(19) United States
(12) Patent Application Publication
(10) Pub. No.: US 2021/0362007 A1
(43) Pub. Date: Nov. 25, 2021

(54) LENS FOR SWIM GOGGLES AND DIVE MASKS

(71) Applicant: FLIPTURN DESIGN, Dallas, TX (US)

(72) Inventors: Mark C. Stephens, Dallas, TX (US); Matthew Buckby, Dallas, TX (US)

(73) Assignee: FLIPTURN DESIGN, Dallas, TX (US)

(21) Appl. No.: 17/300,338

(22) Filed: May 14, 2021

Related U.S. Application Data
(60) Provisional application No. 63/101,902, filed on May 20, 2020.

(51) Int. Cl. A63B 33/00; A61F 9/04; B63C 11/12

(52) U.S. Cl. CPC A63B 33/002; B63C 11/12; A61F 9/04

(57) ABSTRACT

Eye wear of the present invention include a multi-layered lens that include layers formed form different materials or outer layers separated by an inner layer formed from a different material. The multi-layered lens has insulating properties that reduce fogging when used in swimming goggles and face masks.

### RELATED APPLICATION

This application claims priority under 35 U.S.C. § 119(e) from the co-pending U.S. provisional patent application Ser. No. 63/101,902, filed on May 20, 2020, and titled “DOUBLE-PANE VACUUM SEALED LENS FOR SWIMMING GOGGLES AND DIVE MASKS.” The co-pending U.S. provisional patent application Ser. No. 63/101,902, filed on May 20, 2020, and titled “DOUBLE-PANE VACUUM SEALED LENS FOR SWIMMING GOGGLES AND DIVE MASKS” is hereby incorporated by reference.

### FIELD OF THE INVENTION

The invention relates to protective eye wear or face equipment used in an aquatic environment. More particularly, the present invention is directed to a lens used in protective eye wear or face equipment that has insulating properties to reduce fogging.

### BACKGROUND

Goggles and face masks are used for a number of work related activities and sports activities. Goggles and face masks usually have one or more lenses that are secured to a user's head by frame and strap structures. In many cases goggles and face masks includes a frame that secures one or more lenses in front of the user's eyes and creates and isolation chamber or isolation volume between the one or more lenses and the user's eyes and portions of the user's face. When these goggles or face masks are in use, there tends to be environmental differential between the isolation chambers or isolation volumes and the outside of the environments. This environmental differential is such that the moisture and temperature conditions within the isolation chamber or isolation volume are different from the moisture and temperature conditions outside of the isolation chamber or isolation volume, often as a result of the moisture and heat generated by the use's face. The environmental differential described causes the inner surfaces of the one or more lenses within the isolation chamber or isolation volume of the goggle or face masks to fog (fogging) reducing visibility through the one or more lenses.

To reduce fogging on the inner surfaces of the one more lens of the goggle or face mask, de-fogging or anti-fogging coatings can be applied. Such de-fogging or anti-fogging coatings are typically made from a hydrophobic surfactant that provides a thin-film on the inner surfaces of the one or more lenses and thereby helps reduce fogging. De-fogging or anti-fogging coatings have a number of shortcomings. De-fogging or anti-fogging coatings usually need to be reapplied after one or two uses of the goggle or face mask, especially when the goggle or face mask is used in an aquatic environment. De-fogging or anti-fogging coatings can be difficult to apply uniformly and can, therefore, cause distortions and reduce visibility through the one more lenses of the goggle or face mask. Also, de-fogging or anti-fogging coatings can run off of the inner surfaces of the one more lenses of the goggle or face mask and get into the eyes of the user causing irritation and discomfort.

### SUMMARY OF INVENTION

The present invention directed a eye wear used to protect a swimmers eyes while swimming or diving. The eye wear is a pair of goggles or a face mask. The pair of goggles or face mask has one or two lenses. The one or two lenses are secured into a frame structure with a strap structure for securing the eye wear on a user's face and with the one or two lenses in front of the user's eyes. The frame structure and the strap structure are formed from any suitable material or materials but are preferably formed from an elastomer, such as rubber, silicone, soft plastic and combinations thereof.

While the illustrations of lens configurations herein are generally shown as a single lens, these illustrations are provided to describe multi-layered structures of the lenses and the manufacturing of the multi-layered lenses used in the present invention and are not intended to limit the number of lenses or geometric configurations of lenses used.

In accordance with the embodiments of the invention the eye wear has a multi-layered lens structure. The multi-layered lens structures can include layers that are flat, angled, contoured, curved or any combination thereof. The different layers used to form the multi-layered lens can have the same or different thickness and thicknesses can vary within any layer. The multi-layered lens structures includes at least two layers. In use, outer surfaces of an outer layer of the multi-layered lens structure are exposed to an outside environment and inner surfaces of an inner layer of the multi-layered lens structure are exposed to an inside environment within an isolation chamber isolation volume between a user's face the multi-layered lens.

The layers used to form the multi-layered lens are solid, liquid, gas or combinations thereof. Regardless of the materials used to form the multi-layered lens, at least two of the layers are formed from different materials, at least two of the layers are solid and the multi-layered lens reduces transfer of heat or cold (thermally insulates) between the inside surfaces of the multi-layered lens and the outside surfaces of the multi-layered lens.

Where layers of the multi-layered lens are solid, the solid layers are formed from a material selected form, but are not limited to, poly-carbonate, carbon poly-nate, nylon, glass, Polyethylene Terephthalate (PET or PETE or Polyester), High-Density Polyethylene (HDPE), Polyvinyl Chloride (PVC), Low-Density Polyethylene (LDPE), Polypropylene (PP), Polystyrene (PS), tempered glass, and combinations thereof. Where a layer of the multi-layered lens is a gas, the gas layer is formed from a gas selected from, but not limited to, oxygen, nitrogen, xenon, argon, krypton, and mixtures thereof. Where a layer of the multi-layered lens is s liquid layer, the liquid layer is formed from a material selected from, but not limited to, an oil, a gel (such as optically clear silicon gel), a water-based solution and combinations thereof. A layer of the multi-layered lens can also be a vacuum space, as described in detail below.

### BRIEF DESCRIPTION OF THE DRAWINGS

FIGS. 1A-B show schematic representations of cross-sectional views of multi-layered lenses, in accordance with the embodiments of the invention.

FIGS. 2A-B show schematic representations of cross-sectional views of curved or contoured multi-layered lenses, in accordance with the embodiments of the invention.

FIG. 3 shows a view of a pair of swimming goggles with two multi-layered lenses, in accordance with the embodiments of the invention.

FIG. 4 shows a view of a face mask with a multi-layered lens, in accordance with the embodiments of the invention.

### DETAILED DESCRIPTION OF THE INVENTION

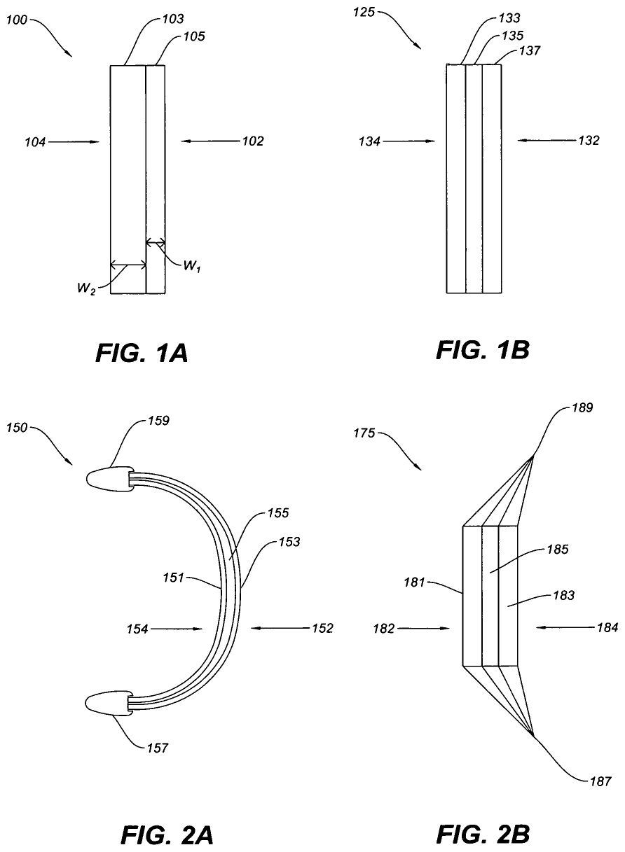

FIG. 1A shows schematic representation of a cross-sectional view of a multi-layered lens 100. The lens 100 has two layers 103 and 105, both of which are optically clear or substantially transparent for viewing through. The layer 103 has a thickness W₂ and the layer 105 has a thickness W₁. In this example the thickness W₂ of the layer 103 is greater than the thickness W₁ of the layer 105 and the layers 103 and 105 are in contact with each other. The layers 103 and 105 are formed from different materials with different heat capacity values or thermal properties that reduce transfer of heat or cold between the surfaces 104 of the layer 103 and the surfaces 102 of the layer 105. The one of the surfaces 102 and 104 corresponds to outside surfaces (outside of the isolation camber or isolation volume of a face mask or goggle) of the multi-layered lens 100 and the other of the surfaces 102 and 104 corresponds to the inside surfaces (within the isolation camber of isolation volume of a face mask or goggle) of the multi-layered lens 100. In this example both of the layers are formed from solid materials such as poly-carbonate, carbon poly-nate, nylon, glass, Polyethylene Terephthalate (PET or PETE or Polyester), High-Density Polyethylene (HDPE), Polyvinyl Chloride (PVC), Low-Density Polyethylene (LDPE), Polypropylene (PP), Polystyrene (PS), tempered glass and combinations thereof.

FIG. 1B shows schematic representation of a cross-sectional views multi-layered lens 125. The multi-layered lens has three layers 133, 135 and 137 that are optically transparent for viewing through. The layers 133, 135 and 137 can have the same thickness or can have varied thickness from each other and varied thickness within any one of the layers 133, 135 and 137, as explained previously. In this example, the outer layers 133 and 137 are formed from solid materials that are the same materials or different materials. Suitable solid materials have been recited previously with reference to FIG. 1A. The inner layer 135 can be solid, liquid, gas or a vacuum space.

Where the inner layer 135 is a solid layer, the center layer 135 is preferably formed from a material that is different form the material used to form the outer layer 133 or the outer layer 137. In accordance with an embodiment of the invention the inner layers a thin film formed form a transparent polymer. Regardless of what material the inner layer 135 is formed form, the inner layer 135 is sandwiched between the two outer layers 133 and 137.

Where the center layer 135 is a gas, the gas can include oxygen, nitrogen, xenon, argon, krypton, and mixtures thereof. Where the center layer 135 is a liquid, the liquid can include an oil, a gel (such as optically clear silicon gel) and a water-based solution and combinations thereof. One of the surfaces 132 and 134 corresponds to outside surfaces of the multi-layered lens 125 and the other of the surfaces 132 and 134 corresponds to the inside surfaces of the multi-layered lens 125. The multi-layered lens 125 reduces the transfer of heat or cold between the surfaces 134 of the layer 133 and the surfaces 132 of the layer 137.

FIG. 2A shows schematic representation of cross-sectional view of a curved and multi-layered lens 150. The multi-layered lens 150 is from two solid curved outer layers 151 and 153 with a solid, liquid or gas inner layer 155, therebetween. Alternatively, the multi-layered lens 150 is from two solid curved outer layers 151 and 153 with a vacuum space 155, therebetween. Suitable, solid, gas and liquid materials are recited above with reference to FIGS. 1A-B.

As described previously, the two solid curved outer layers 151 and 153 can be formed from the same or different materials. In accordance with an embodiment of the invention the solid curved outer layers 151/153 and the inner layer 155 are secured together through end pieces 157 and 159. The end pieces 157 and 159 hermetically seal (encapsulate) the inner layer 155 from exposure to an outside environment. The end pieces 157 and 159 typically encircles entire edges of the multi-layered lens 150. The end pieces 157 and 159 can, for examples be a monolithic (formed from a single piece) gasket or can be formed in parts. The end pieces 157 and 159 can be formed from an adhesive or glues and any other number of materials suitable for sealing the inner layer 155 between the two solid curved outer layers 151 and 153.

The concave surfaces 154 of the curved solid outer layer 151 correspond to inside surfaces of the multi-layered lens 150 and the convex surfaces 152 of the solid curved outer layer 153 correspond to outside surfaces of the multi-layer lens 150. The multi-layered lens 150 reduces the transfer of heat or cold between the surfaces 154 of the solid curved outer layer 151 and the surfaces 152 of the solid curved outer layer 153.

FIG. 2B shows schematic representation of cross-sectional view of an angled or contoured multi-layered lens 175. The multi-layered lens 175 is from two solid outer layers 181 and 183 with a solid, liquid or gas inner layer 185, therebetween. Alternatively the angled or contoured multi-layered lens 175 is from two solid outer layers 181 and 183 with a vacuum space 185, therebetween. This example shows that two solid outer layers 181 and 183 and the inner layer 185 have varied thickness and tapper to the ends 187 and 189.

As described previously the two solid outer layers 181 and 183 can be formed from the same or different materials. In accordance with an embodiment of the invention two solid outer layers 181/183 and the inner layer 185 are sealed at tapered ends 187 and 189. The tapered ends 187 and 189 typically surround the entire edge of the multi-layered lens 175 and hermetically seal (encapsulate) the inner layer 185 from exposure to an outside environment. The concave surfaces 184 of the solid outer layer 183 correspond to inside surfaces of the multi-layered lens 175 and the convex surfaces 182 of the solid outer layer 181 correspond to outside surfaces of the multi-layer lens 175. The multi-layered lens 175 reduces the transfer of heat or cold between the surfaces 184 of the solid outer layer 183 and the surfaces 182 of the solid outer layer 181.

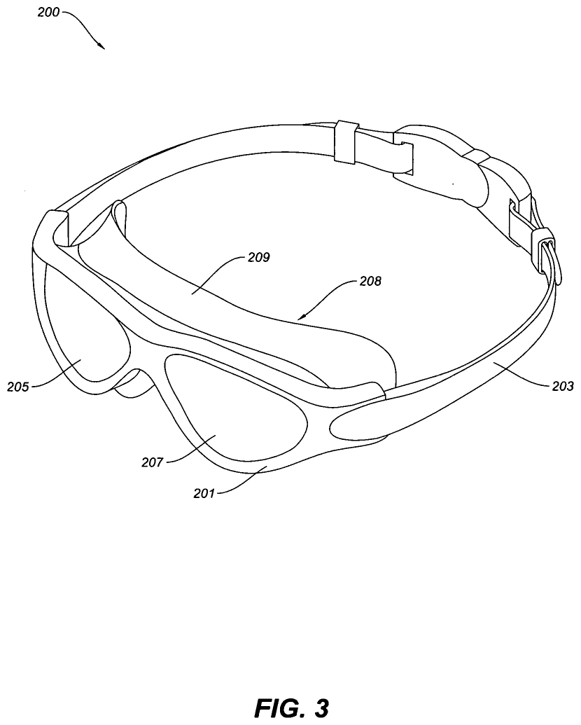

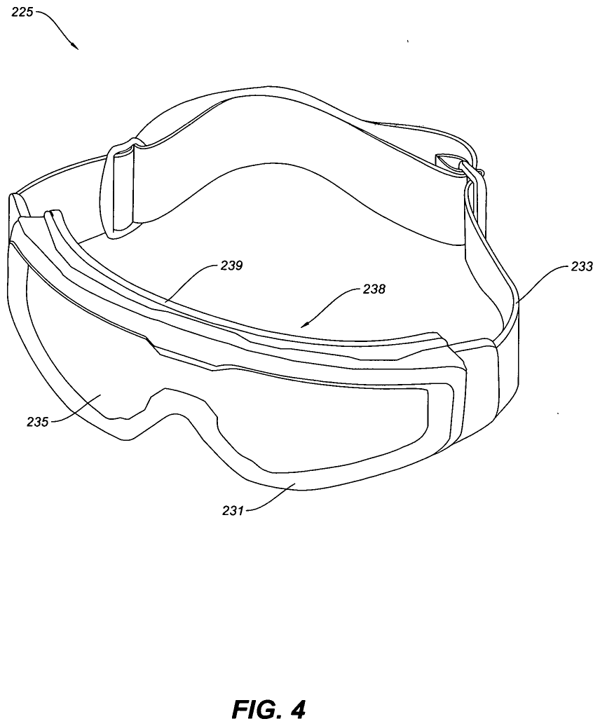

FIG. 3 shows a view of a pair of swimming goggles 200 with two multi-layered lenses 205 and 207. The two multi-layered lenses 205 and 207 have any number of configurations, such as described with reference to FIGS. 1A-B and FIGS. 2A-B above. The pair of swimming goggles 200 includes a frame structure 201 for holding the two multi-layered lenses 205 and 207 and a skirt structure 209 for securing the pair of goggles 200 against a user's face through a strap 203 and, thereby, creating an isolation chamber or isolation volume 208 between the user's face with the multi-layered lenses 205 and 207 positioned in front the user's eyes. The multi-layered lenses 205 and 207 reduce the transfer of heat or cold between outer lens surfaces and inner lens surfaces and within the isolation chamber or isolation volume 208 to thereby reduce fogging. The frame structure 201, the skirt structure 209 strap are typically made from a soft elastomeric material, such as silicone, rubber and soft plastic.

FIG. 4 shows a view of a face mask 225 with a multi-layered lens 235. The multi-layered lens 235 has any number of configurations, such as described with reference to FIGS. 1A-B and FIGS. 2A-B, above. The face mask 225 includes a frame structure 231 for holding the multi-layered lens 235 and a skirt structure 239 and strap 233 for securing the face mask 225 against a user's face and, thereby, create an isolation chamber or isolation volume 238 between the user's face with the multi-layered lens 235 positioned in front the user's eyes. The multi-layered lens 235 reduces the transfer of heat or cold between outer surfaces and inner surfaces and within the isolation chamber or isolation volume 238 to thereby reduce fogging. The frame structure 231 the skirt structure 239 and the strap 233 are typically made for soft elastomeric material, such as silicone, rubber and soft plastic.

The present invention has been described in terms of specific embodiments incorporating details to facilitate the understanding of the principles of construction and operation of the invention. For example, the multi-layered lens (FIGS. 1A-B and FIGS. 2A-B) and the eye wear (FIGS. 3-4) can have any number of shapes. As such, references herein to specific embodiments and details thereof are not intended to limit the scope of the claims appended hereto.

What is claimed is:

1. Eye wear comprising at least one a multi-layered lens comprising two solid outside layers that encapsulate an inner layer, wherein the inner layer is a liquid layer, a gas layer or a vacuum space.

2. Eye wear of claim 1, wherein the two solid outside layers are formed form one or more of poly-carbonate, carbon poly-nate, nylon, glass, Polyethylene Terephthalate (PET or PETE or Polyester), High-Density Polyethylene (HDPE), Polyvinyl Chloride (PVC), Low-Density Polyethylene (LDPE), Polypropylene (PP), Polystyrene (PS) and tempered glass.

3. Eye wear of claim 1, wherein the inner layer is formed from one or more of an oil, a gel (such as optically clear silicon gel) and a water-based solution.

4. Eye wear of claim 1, wherein the inner layer is formed from one or more of oxygen, nitrogen, xenon, argon, krypton.

5. Eye wear of claim 1, further comprising a frame, a skirt and a strap and wherein the eye wear is a pair of goggle or a face mask.

6. Eye wear comprising one or more multi-layered lenses with three layers that include two solid outside layers and a solid inner layer, wherein the solid inner layer is formed form a material that is a different from the material used to form the two solid outside layers.

7. Eye wear of claim 6, wherein the two solid outside layers are formed form one or more of poly-carbonate, carbon poly-nate, nylon, glass, Polyethylene Terephthalate (PET or PETE or Polyester), High-Density Polyethylene (HDPE), Polyvinyl Chloride (PVC), Low-Density Polyethylene (LDPE), Polypropylene (PP), Polystyrene (PS) and tempered glass.

8. Eye wear of claim 6, wherein inner is a thin film formed form a transparent polymer.

9. Eye wear of claim 6, wherein one or more of the solid outside layers and the solid inner layer have varying thicknesses.

10. Eye wear of claim 6, further comprising a frame with a skirt and a strap to create an isolation chamber or isolation volume between the one or more multi-layered lenses and a user's face.

11. Eye wear comprising multi-layered lenses comprising two solid outside layers a frame with a skirt and a strap to create an isolation chamber or isolation volume between the multi-layered lenses and a user's face.
